(19) United States
(12) Patent Application Publication
(10) Pub. No.: US 2025/0237646 A1
(43) Pub. Date: Jul. 24, 2025

(54) CREATININE LATERAL FLOW ASSAY DEVICES AND METHODS OF PRODUCTION AND USE THEREOF

(71) Applicant: Siemens Healthcare Diagnostics Inc., Tarrytown, NY (US)

(72) Inventors: Aaron Kauffmann, Elkhart, IN (US); Jeffrey Mayfield, South Bend, IN (US)

(73) Assignee: Siemens Healthcare Diagnostics Inc., Tarrytown, NY (US)

(21) Appl. No.: 18/853,945

(22) PCT Filed: Apr. 10, 2023
(86) PCT No.: PCT/US2023/018015

§ 371 Date: Oct. 3, 2024

Related U.S. Application Data
(60) Provisional application No. 63/363,042, filed on Apr. 15, 2022.

(51) Int. Cl. G01N 33/543; G01N 33/70

(52) U.S. Cl. CPC G01N 33/54388; G01N 33/70; G01N 2333/46; G01N 2800/22

(57) ABSTRACT

Lateral flow assay devices and kits for use in the detection of creatinine and in the detection of hemolysis in a biological fluid sample are disclosed. Methods of making and using the lateral flow assay devices and kits are also disclosed.

### CROSS-REFERENCE TO RELATED APPLICATIONS

This application claims benefit under 35 USC § 119(e) of U.S. Provisional Application No. 63/363,042, filed Apr. 15, 2022. The entire contents of the above-referenced patent application(s) are hereby expressly incorporated herein by reference.

### BACKGROUND

Hemolysis refers to the destruction or dissolution of red blood cells (RBCs) which results in the release of hemoglobin (“free hemoglobin”) into surrounding liquid. In the case of a whole blood sample, the free hemoglobin is released into the surrounding plasma. In the case of urine, the free hemoglobin is released into the surrounding water. The occurrence of hemolyzed RBCs may be the result of a patient's medical condition or by the mishandling the sample itself.

Hemolysis is a preanalytical error of concern when testing patient's samples. When severe enough, hemolysis may result in inaccurate laboratory test results. For example, it is known that hemolysis may falsely increase magnesium, ammonia, phosphorus, glucose, calcium, iron, albumin, amylase, lipase, lactate dehydrogenase (LDH), creatine kinase (CK), aspartate transaminase (AST), alanine transaminase (ALT), LDH, total bilirubin, total protein, and hemoglobin levels. In addition, it is known that hemolysis may falsely decrease potassium, creatinine, and alkaline phosphatase levels.

The detection of hemolysis in whole blood samples has traditionally been difficult and time consuming. In a central laboratory setting, a whole blood sample is subjected to centrifugation which generates plasma that is interrogated optically either in the near-infrared (NIR) or visible wavelength regions. While this technique is very effective, it is both complex and time consuming—thereby making this technique ineffective for Point of Care (POC) applications.

In the point of care arena, some systems detect hemolysis electrochemically. However, electrochemical detection of hemoglobin and hematocrit is known to be inaccurate; in addition, hemolysis would only be detected after the electrochemical reagents are consumed, which could waste sample, reagent, and time.

It is important to run whole blood samples as quickly as possible after draw; any delays can impact the sample result. In addition, the samples must be aliquoted and centrifuged before a hemolysis measurement can be made. The requirement for centrifugation, however, limits the settings in which a hemolysis measurement can be determined, and this requirement necessarily lengthens the time between sample collection and testing.

Before a patient can have an iodine, boron, or similar dye compound injected for a contrast or angiographic CT scan, the patient must be checked for a pre-existing decreased kidney function, as decreased kidney function is a risk factor for contrast-induced nephropathy. One of the measurements utilized to estimate risk is the level of creatinine in serum. There are a few Point-Of-Care (POC) whole blood creatinine tests currently available; however, these are all based on potentiometric assays. Currently, there are no POC whole blood creatinine tests based on visual or optical detection assays.

Therefore, there is a need in the art for new and improved lateral flow assay devices and methods that allow for detection of at least one target analyte (such as, but not limited to, creatinine) in combination with detection of hemolysis in biological fluid samples.

### BRIEF DESCRIPTION OF THE DRAWINGS

FIG. 1 is a top plan view of one non-limiting embodiment of a lateral flow cartridge constructed in accordance with the present disclosure.

FIG. 2 is a cross-sectional view of the lateral flow cartridge of FIG. 1.

FIG. 3 contains a flow chart illustrating the steps involved in a method of use of the lateral flow cartridge of FIGS. 1-2.

FIG. 4 is a top plan view of another non-limiting embodiment of a lateral flow cartridge constructed in accordance with the present disclosure.

FIG. 5 is a cross-sectional view of the lateral flow cartridge of FIG. 4.

FIG. 6 depicts the colorimetric detection of creatinine using a lateral flow assay constructed in accordance with the present disclosure.

FIG. 7 graphically illustrates RGB values from the images of FIG. 6.

### DETAILED DESCRIPTION

Before explaining at least one embodiment of the present disclosure in detail by way of exemplary language and results, it is to be understood that the present disclosure is not limited in its application to the details of construction and the arrangement of the components set forth in the following description. The present disclosure is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions/devices, kits, and/or methods disclosed herein can be made and executed without undue experimentation in light of the present disclosure. While the compositions/devices, kits, and/or methods have been described in terms of particular embodiments, it will be apparent to those of skill in the art that variations may be applied to the compositions/devices, kits, and/or methods and in the steps or in the sequence of steps of the methods described herein without departing from the concept, spirit, and scope of the present disclosure. All such similar substitutions and modifications apparent to those skilled in the art are deemed to be within the spirit, scope, and concept of the present disclosure as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

The use of ordinal number terminology (i.e., “first,” “second,” “third,” “fourth,” etc.) is solely for the purpose of differentiating between two or more items and, unless explicitly stated otherwise, is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps. For example, a process, method, article, or apparatus that comprises a list of elements is not necessarily limited to only those elements but may include other elements not expressly listed or inherently present therein.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrase “associated with” includes both direct association of two moieties to one another as well as indirect association of two moieties to one another. Non-limiting examples of associations include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety.

The term “liquid test sample” or “liquid biological sample” as used herein will be understood to include any type of biological fluid sample that may be utilized in accordance with the present disclosure. Examples of biological fluid samples that may be utilized include, but are not limited to, whole blood or any portion thereof (i.e., plasma or serum), saliva, sputum, cerebrospinal fluid (CSF), intestinal fluid, intraperitoneal fluid, cystic fluid, sweat, interstitial fluid, tears, mucus, urine, bladder wash, semen, combinations thereof, and the like. The volume of the liquid test sample utilized in accordance with the present disclosure may be (for example but not by way of limitation) from about 0.1 μl to about 100 μl.

As used herein, the term “volume” as it relates to the liquid test sample utilized in accordance with the present disclosure means from about 0.1 μl to about 100 μl, or from about 1 μl to about 75 μl, or from about 2 μl to about 60 μl, or less than or equal to about 50 μl.

The term “patient” includes human and veterinary subjects. In certain embodiments, a patient is a mammal. In certain other embodiments, the patient is a human. “Mammal” for purposes of diagnosis/treatment refers to any animal classified as a mammal, including human, domestic and farm animals, nonhuman primates, and zoo, sports, or pet animals, such as dogs, horses, cats, cows, etc.

Turning now to particular non-limiting embodiments of the inventive concept(s), the present disclosure is related to creatinine lateral flow assay devices (as well as kits containing same) that can be utilized in methods of determining the presence and/or concentration of creatinine in a biological fluid sample. The lateral flow assay devices of the present disclosure also allow for the detection of hemolysis in the biological fluid sample.

Certain non-limiting embodiments of the present disclosure include a lateral flow assay device that comprises lateral flow assay device for detection of creatinine in a biological fluid sample. The lateral flow assay device includes a housing, a lateral flow membrane, at least two filter membranes, and at least one assay reagent.

The housing comprises an upper surface, a lower surface, a first end, a second end, and an interior space extending between the upper and lower surfaces and the first and second ends. The housing further comprises at least a first well and a second well that each extend through the upper surface of the housing into the interior space and that are spaced apart from one another. In addition, the housing comprises a sample reservoir extending through the upper surface of the housing upstream from the first and second wells.

The lateral flow membrane is disposed in the interior space of the housing and defines a path for fluid flow; the lateral flow membrane is in fluidic communication with the sample reservoir and the first and second wells. In addition, the lateral flow membrane has an upper surface and a lower surface. The lateral flow assay device also includes a first filter membrane disposed in the first well of the housing and on a portion of the upper surface of the lateral flow membrane such that the first filter membrane is in fluidic communication with the lateral flow membrane; in addition, at least one assay reagent is embedded in the first filter membrane, wherein the at least one assay reagent detects creatinine. The lateral flow assay device further includes a second filter membrane disposed in the second well of the housing and on a portion of the upper surface of the lateral flow membrane such that the second filter membrane is in fluidic communication with the lateral flow membrane. As such, the presence and/or concentration of creatinine is detected in the first well of the lateral flow assay device, and hemolysis is detected in the second well.

Any types of assay reagents known in the art or otherwise contemplated herein that are capable of being embedded in a filter membrane and detecting creatinine via a lateral flow assay and visual/optical detection method may be utilized in accordance with the present disclosure. It is contemplated that virtually any reagent used in the fields of biological, chemical, or biochemical analyses and assays could be used in the lateral flow assay device. It is contemplated that these reagents may undergo physical and/or chemical changes when bound to an analyte of interest whereby the intensity, nature, frequency, or type of signal generated by the reagent-analyte complex is directly proportional or inversely proportional to the concentration of the analyte existing within the fluid sample. These reagents may contain indicator dyes, metals, enzymes, polymers, antibodies, and/or chemicals that, when reacting with an analyte(s) of interest, may exhibit a change in color.

In certain particular (but non-limiting) embodiments, the at least one assay reagent includes a colorimetric reagent.

In certain particular (but non-limiting) embodiments, the at least one assay reagent can comprise 3,5-dinitrobenzoic acid and sodium hydroxide. In another particular (but non-limiting) embodiment, the at least one assay reagent is enzymatic in nature.

The lateral flow membrane and first and second filter membranes may be formed of any membrane materials known in the art or otherwise contemplated herein, so long as the lateral assay device formed therefrom can function in accordance with the present disclosure.

For example, the lateral flow membrane may be formed of a cellulose membrane, such as, but not limited to, a nitrocellulose or carboxymethyl cellulose membrane, as are commercially available in the art. Also, for example (but not by way of limitation), one or both of the first and second filter membranes may be formed of a plasma separation membrane as are commercially available in the art. In certain particular (but non-limiting) embodiments, the plasma separation membrane comprises an asymmetric material which is able to retain a plurality of whole blood cells thereon while allowing plasma and small molecules/complexes to travel there through. A number of different plasma separation membranes are commercially available and may be suitable for use as the first and/or second filter membranes. For example (but not by way of limitation), the first and/or second filter membranes may comprise an asymmetric cellulose filter membrane (such as, but not limited to, an asymmetric nitrocellulose filter membrane), starch, carboxymethylcellulose, and/or glass fiber. Alternatively, the plasma separation membrane may comprise an asymmetric polysulfone material as is commercially available from Pall Corporation (currently sold under the trademark Vivid™).

The plasma separation membranes utilized in certain non-limiting embodiments may be capable of separating plasma in the first and/or second filter membranes. Alternatively, the first and/or second filter membrane may comprise at least one red blood cell (RBC) agglutinating protein, an anti-RBC antibody, and/or potato lectin embedded therein to further assist in separation so that plasma can continue flowing through the separation membrane.

In certain particular (but non-limiting) embodiments, the lateral flow assay device further comprises at least one additional filter membrane disposed in the interior space of the housing downstream of the second well and in fluidic communication with the lateral flow membrane; in this manner, the at least one additional filter membrane functions to ensure flow of the biological fluid sample through the various wells of the lateral flow assay device and also serve as a waste receptacle to absorb excess sample.

The lateral flow assay devices of the present disclosure may be designed and configured for manual use by an individual. In this manner, the color change at each of the wells is visually detected by the individual via a visual comparison to a reference device containing a plurality of reference colors which correspond to different levels of creatine, hematocrit, or hemolysis. Alternatively, the lateral flow assay device may be designed and configured as a cartridge for insertion within a clinical analyzer instrument that detects each of the color changes at the various wells/detection sites.

While a particular configuration of wells is described herein above, it will be understood that the order of wells (and thus the detection of hematocrit, creatinine, and hemolysis) may be changed, as required by the membranes and/or reagents utilized. As such, the scope of the present disclosure includes changes to the order and sequence of steps performed by the lateral flow assay devices disclosed herein. In addition, the scope of the present disclosure includes lateral flow assay devices that include fewer than the three wells described above, as well as the lateral flow assay devices that include one or more additional wells for performing one or more additional target analyte detection assays at the same time as the hematocrit, creatinine, and/or hemolysis assays on the same biological fluid sample.

For example, certain non-limiting embodiments of the present disclosure are directed to a lateral flow assay device similar to the device described herein above, except that in these embodiments, the lateral flow assay device has three wells for detection of hematocrit in addition to creatinine and hemolysis. As such, the housing of the device includes at least a first well, a second well, and a third well that each extend through the upper surface of the housing into the interior space and that are spaced apart from one another and downstream of the sample reservoir, and the lateral flow membrane is in fluidic communication with the sample reservoir and the first, second, and third wells. Then the lateral flow assay device is further defined as including a first filter membrane disposed in the first well of the housing and on a portion of the upper surface of the lateral flow membrane such that the first filter membrane is in fluidic communication with the lateral flow membrane; a second filter membrane disposed in the second well of the housing and on a portion of the upper surface of the lateral flow membrane such that the second filter membrane is in fluidic communication with the lateral flow membrane; at least one assay reagent embedded in the second filter membrane, wherein the at least one assay reagent detects the presence and/or concentration of creatinine; and a third filter membrane disposed in the third well of the housing and on a portion of the upper surface of the lateral flow membrane such that the third filter membrane is in fluidic communication with the lateral flow membrane. In this manner, a hematocrit measurement is detected in the first well, the presence and/or concentration of creatinine is detected in the second well, and hemolysis is detected in the third well.

The various components of the lateral flow assay device may be as described herein above with respect to the previously described embodiments. In addition, if at least one additional filter membrane is present in the device to absorb excess sample, the additional filter membrane is disposed downstream of the third well.

The device(s), kit(s), and method(s) disclosed or otherwise contemplated herein may be used for the analysis of any liquid test sample, including, without limitation, whole blood, plasma, serum, or urine. In certain non-limiting embodiments, the biological fluid sample is a whole blood sample which includes a quantity of whole blood cells, including red blood cells, white blood cells, and platelets. Within the sample, the extent of hemolysis may correlate to an amount of hemoglobin therein. As used herein, it is understood that the term “hemoglobin” refers to any and all hemoglobin molecules obtained either from drawn blood or by recombinant procedures in their oxygenated, deoxygenated, dimeric, tetrameric, or various polymerized forms. Hemoglobin is commonly known as the oxygen-carrying pigment and predominant protein of red blood cells. Hemoglobin is composed of four protein chains, two alpha chains and two beta chains, each with a ring-like heme group containing an iron atom. Oxygen binds reversibly to these iron atoms. In its oxygenated state, hemoglobin may be referred to as oxyhemoglobin and is characterized by a bright red. In the reduced state, hemoglobin may be referred to as deoxyhemoglobin and is characterized by a purple-blue color.

Certain non-limiting embodiments of the present disclosure are directed to kits useful for conveniently performing an assay for the determination of a concentration of creatinine. These kits include one or more of any of the lateral flow assay devices disclosed or otherwise contemplated herein above, either alone or in combination with other assay reagent(s) and/or component(s) for conducting any of the particular assays described or otherwise contemplated herein. In particular (but not by way of limitation), the kits of the present disclosure may further contain one or more other component(s) or reagent(s) for performing biological fluid sample collection(s) and/or diagnostic application(s) in accordance with the present disclosure. For example (but not by way of limitation), the kits may include one or more biological fluid sample collection device(s), one or more assay reagent(s), one or more calibration reagent(s), one or more quality control reagent(s), one or more wash reagent(s), etc. The nature of these additional component(s)/reagent(s) will depend upon various factors such as (but not limited to) the type of biological fluid sample and the diagnostic assay format, and identification thereof is well within the skill of one of ordinary skill in the art; therefore, no further description thereof is deemed necessary.

Also, the various components/reagents present in the kit may each be in separate containers/compartments, or various components/reagents can be combined in one or more containers/compartments, depending on the cross-reactivity and stability of the components/reagents. The kit can further include other separately packaged reagents for conducting an assay.

In certain particular (but non-limiting) embodiments, the lateral flow assay device(s) present in the kit is disposed in an airtight foiled package in which the lateral flow assay device is stored. When multiple lateral flow assay devices are present, each individual device may be stored in a separate airtight foiled package.

The relative amounts of the various components/reagents present in the kits can vary widely to provide for concentrations of the components/reagents that substantially optimize the reactions that need to occur during the assay methods and further to optimize substantially the sensitivity of an assay. Positive and/or negative controls may be included with the kit. The kit can further include a set of written instructions explaining how to use the kit. A kit of this nature can be used in any of the methods described or otherwise contemplated herein.

Certain non-limiting embodiments of the present disclosure are directed to a method for detection of creatinine in a biological fluid sample. The method comprises the steps of: (1) dispensing the biological fluid sample into the sample reservoir of any of the lateral flow assay devices disclosed or otherwise contemplated herein that has at least two wells; (2) allowing the biological fluid sample to flow through the lateral flow assay device; (3) measuring a signal generated in each of the first and second wells of the lateral flow assay device; (4) detecting the presence and/or concentration of creatinine based on the signal generated in the first well; and (5) determining if hemolysis is present based on the signal generated in the second well.

One or more of the steps of the method may be performed manually by an individual (such as, but not by way of limitation, through the use of a color chart containing various shades/intensities of potential color for each well so as to detect the presence and/or concentration of creatinine as well as the presence and/or amount of hematocrit and/or hemolysis present). Alternatively (and/or in addition thereto), the method may further include the step of inserting the lateral flow assay device into a clinical analyzer instrument after step (1) (and before or after step (2)); in this manner, steps (3), (4), and (5) are performed by the clinical analyzer instrument. For example (but not by way of limitation), the method may be utilized with the Atellica or ADVIA® Clinical Chemistry Analyzer Systems (Siemens Medical Solutions USA, Inc., Malvern, PA) or with any other clinical chemistry analyzer instrument capable of performing a lateral flow assay and analyzing colorimetric results produced therefrom.

Certain non-limiting embodiments of the present disclosure are directed to a method for detection of the presence and/or concentration of creatinine in a biological fluid sample. The method comprises the steps of: (1) dispensing the biological fluid sample into the sample reservoir of any of the lateral flow assay devices disclosed or otherwise contemplated herein that has at least three wells; (2) allowing the biological fluid sample to flow through the lateral flow assay device; (3) measuring a signal generated in each of the first, second, and third wells of the lateral flow assay device; (4) detecting a hematocrit value based on the signal generated in the first well; (5) detecting the presence and/or concentration of creatinine based on the signal generated in the second well; and (6) determining if hemolysis is present based on the signal generated in the third well.

One or more of the steps of the method may be performed manually by an individual (such as, but not by way of limitation, through the use of a color chart containing various shades/intensities of potential color for each well so as to detect the presence and/or concentration of creatinine as well as the presence and/or amount of hematocrit and/or hemolysis present). Alternatively (and/or in addition thereto), the method may further include the step of inserting the lateral flow assay device into a clinical analyzer instrument after step (1) (and before or after step (2)); in this manner, steps (3), (4), (5), and (6) are performed by the clinical analyzer instrument. For example (but not by way of limitation), the method may be utilized with the Atellica or ADVIA® Clinical Chemistry Analyzer Systems (Siemens Medical Solutions USA, Inc., Malvern, PA) or with any other clinical chemistry analyzer instrument capable of performing a lateral flow assay and analyzing colorimetric results produced therefrom.

### EXAMPLES

Examples are provided hereinbelow. However, the present disclosure is to be understood to not be limited in its application to the specific experimentation, results, and laboratory procedures disclosed herein after. Rather, the Examples are simply provided as one of various embodiments and is meant to be exemplary, not exhaustive.

### Example 1

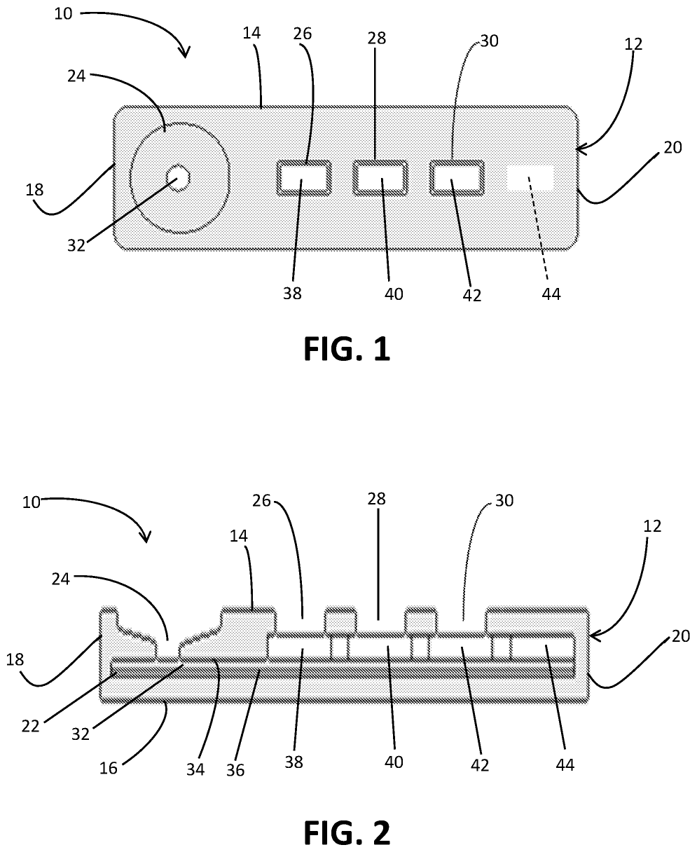

FIGS. 1-2 depict one non-limiting embodiment of a lateral flow assay device 10 for detection of creatinine and constructed in accordance with the present disclosure. The lateral flow assay device 10 includes a housing 12 that comprises an upper surface 14, a lower surface 16, a first end 18, a second end 20, and an interior space 22 extending between the upper and lower surfaces 14 and 16 and the first and second ends 18 and 20. The housing 12 further comprises a sample reservoir 24 and a first well 26, a second well 28, and a third well 30. Each of the sample reservoir 24 and the wells 26, 28, and 30 are spaced apart from one another and extend through the upper surface 14 of the housing 12 into the interior space 22. The sample reservoir 24 is in relatively close proximity to the first end 18 of the housing 12, and each of the wells 26, 28, and 30 is positioned downstream of the sample reservoir 24.

A lateral flow membrane 32 is disposed in the interior space 22 of the housing 12 and defines a path for fluid flow. The lateral flow membrane 32 is in fluidic communication with the sample reservoir 24 and the first, second, and third wells 26, 28, and 30. In addition, the lateral flow membrane 32 has an upper surface 34 and a lower surface 36. The lateral flow assay device 10 also includes a first filter membrane 38 disposed in the first well 26 of the housing 12 and on a portion of the upper surface 34 of the lateral flow membrane 32 such that the first filter membrane 38 is in fluidic communication with the lateral flow membrane 32. The lateral flow assay device 10 also includes a second filter membrane 40 disposed in the second well 28 of the housing 12 and on a portion of the upper surface 34 of the lateral flow membrane 32 such that the second filter membrane 40 is in fluidic communication with the lateral flow membrane 32; also, at least one assay reagent for detection of creatinine is embedded in the second filter membrane 40. The lateral flow assay device 10 further includes a third filter membrane 42 disposed in the third well 30 of the housing 12 and on a portion of the upper surface 34 of the lateral flow membrane 32 such that the third filter membrane 42 is in fluidic communication with the lateral flow membrane 32. As such, hematocrit is detected in the first well 26, the presence and/or concentration of creatinine is detected in the second well 28, and hemolysis is detected in the third well 30 of the lateral flow assay device 10.

In addition, the lateral flow assay device 10 also includes an additional filter membrane 44 disposed in the interior space 22 of the housing 12 downstream of the third well 30 and in fluidic communication with the lateral flow membrane 32; in this manner, the additional filter membrane 44 functions to ensure flow of the biological fluid sample through the wells 26, 28, and 30 of the lateral flow assay device 10 and also serves as a waste receptacle to absorb excess sample.

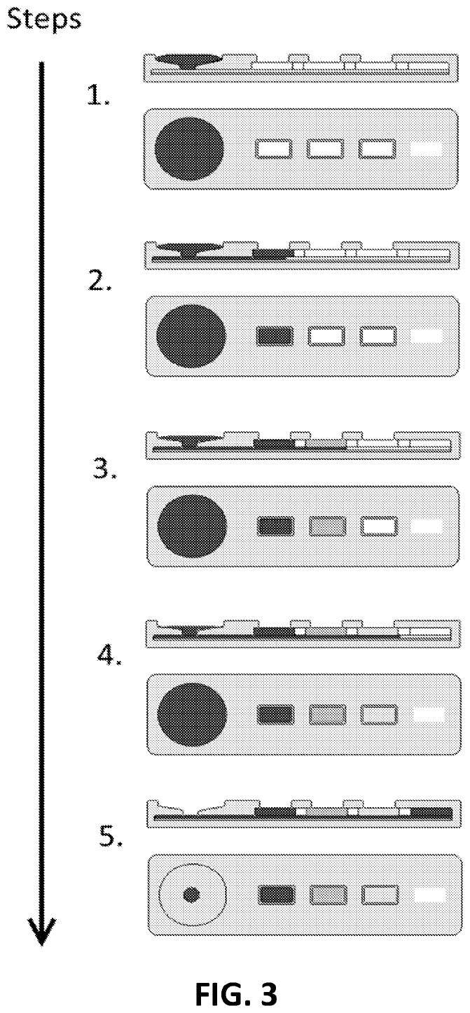

FIG. 3 illustrates use of the cartridge depicted in FIGS. 1-2 for the detection of creatinine. In step (1), blood is added to the sample reservoir. In step (2), blood saturates the first cellulose filter membrane in the first well for a hematocrit estimate. In step (3), plasma passes through the lateral flow membrane, and creatinine reacts with the reagents (such as, but not limited to, 3,5-dinitrobenzoic acid and sodium hydroxide) embedded in the second filter membrane for detection of creatinine in the second well. In step (4), plasma passes through the third lateral flow membrane, and any hemolysis is presented in the third well. In step (5), excess sample is drawn into the fourth filter membrane downstream of the three wells/membranes.

While FIGS. 1-3 depict the flow of sample through the lateral flow assay device in a sequential fashion, such that the sample travels through the individual wells in sequence, it will be understood that the scope of the present disclosure also includes lateral flow assay devices in which the wells are arranged in parallel, so that the sample travels through the wells in parallel (i.e., substantially simultaneously). Therefore, the scope of the present disclosure includes a lateral flow assay device wherein the sample reservoir is disposed upstream of and substantially equidistant from each of the wells, and wherein the lateral flow membrane defines paths for fluid flow that extend from the sample reservoir to each of the wells. In addition, the scope of the present disclosure includes a lateral flow assay device comprising multiple sample reservoirs planarly disposed to one another and that are each disposed upstream of one or more wells; in this manner, each of the multiple sample reservoirs is disposed upstream of the well(s) at substantially the same distance as the distance between the other sample reservoir(s) and well(s).

### Example 2

This Example describes another non-limiting embodiment of a lateral flow assay device similar to that disclosed in Example 1, except that the lateral flow assay device only contains two wells for the detection of creatinine and hemolysis.

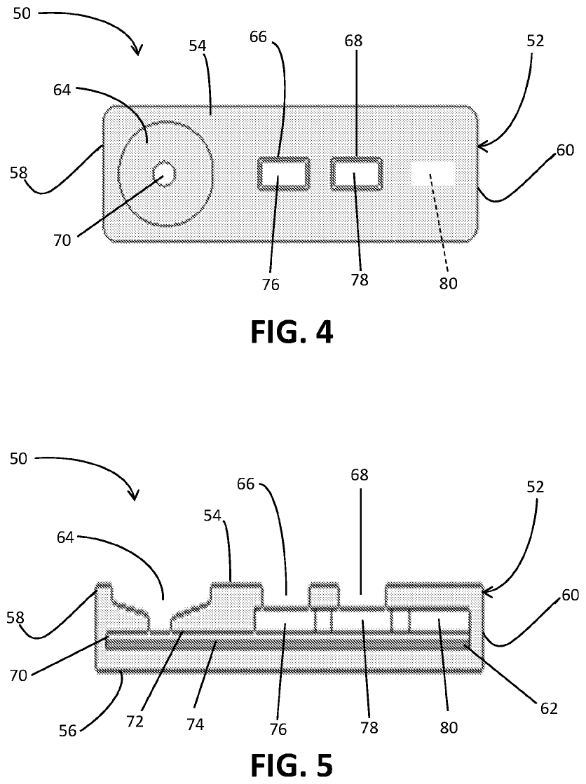

FIGS. 4-5 depict a lateral flow assay device 50 for detection of creatinine and constructed in accordance with the present disclosure. The lateral flow assay device 50 includes a housing 52 that comprises an upper surface 54, a lower surface 56, a first end 58, a second end 60, and an interior space 62 extending between the upper and lower surfaces 54 and 56 and the first and second ends 58 and 60. The housing 52 further comprises a sample reservoir 64, a first well 66, and a second well 68. Each of the sample reservoir 64 and the wells 66 and 68 are spaced apart from one another and extend through the upper surface 54 of the housing 52 into the interior space 62. The sample reservoir 64 is in relatively close proximity to the first end 58 of the housing 52, and each of the wells 66 and 68 is positioned downstream of the sample reservoir 64.

A lateral flow membrane 70 is disposed in the interior space 62 of the housing 52 and defines a path for fluid flow. The lateral flow membrane 70 is in fluidic communication with the sample reservoir 64 and the first and second wells 66 and 68. In addition, the lateral flow membrane 70 has an upper surface 72 and a lower surface 74. The lateral flow assay device 50 also includes a first filter membrane 76 disposed in the first well 66 of the housing 52 and on a portion of the upper surface 72 of the lateral flow membrane 70 such that the first filter membrane 76 is in fluidic communication with the lateral flow membrane 70; also, at least one assay reagent for detection of creatinine is embedded in the first filter membrane 76. The lateral flow assay device 50 further includes a second filter membrane 78 disposed in the second well 68 of the housing 52 and on a portion of the upper surface 72 of the lateral flow membrane 70 such that the second filter membrane 78 is in fluidic communication with the lateral flow membrane 70. As such, the presence and/or concentration of creatinine is detected in the first well 66, and hemolysis is detected in the second well 68 of the lateral flow assay device 50.

In addition, the lateral flow assay device 50 also includes an additional filter membrane 80 disposed in the interior space 62 of the housing 52 downstream of the second well 68 and in fluidic communication with the lateral flow membrane 70; in this manner, the additional filter membrane 80 functions to ensure flow of the biological fluid sample through the wells 66 and 68 of the lateral flow assay device 50 and also serves as a waste receptacle to absorb excess sample.

### Example 3

This Example demonstrates use of the lateral flow assay devices constructed in accordance with the present disclosure to detect creatinine.

A biological fluid sample is obtained from a finger stick or venous draw. A lateral flow assay device as described or otherwise contemplated herein is removed from an airtight foiled package, and a 10-100 μl volume of the biological fluid sample is placed in the sample reservoir of the lateral flow assay device. The sample is drawn along the lateral flow membrane and drawn into the at least three separate filter membranes placed on the top of the lateral flow membrane. The filters include at least a first membrane, two asymmetric membranes, optionally RBC agglutinating protein bound filters, and a generic filter at the end to absorb excess sample.

The first filter has no reagent embedded therein and is used for obtaining a hematocrit value for the sample in a first well of the device. The second filter is an asymmetric membrane that is embedded with a colorimetric reagent (such as, but not limited to, NaOH and 3,5-dinitrobenzoic acid) for detection of the analyte of interest (i.e., creatinine) in a second well of the device. The third filter is also an asymmetric membrane, has no reagent embedded therein, and is used for obtaining a signal generated by background hemolysis in a third well of the device. In addition, the lateral flow assay device may further include one or more additional membrane/reagent/well combinations for performing one or more additional target analyte detection assays in addition to the detections of hematocrit, creatinine, and hemolysis.

The amount of creatinine present is determined as follows. A reference white balance is taken from the end of the lateral flow assay device's housing. The hemolysis background is taken from the third well, and this value is subtracted from the signal obtained from the second well.

Plasma volume can be metered by the asymmetric membrane's size.

Precision of the assay may be improved by extended exposures to reduce noise, increasing the filter membrane sizes and hence area imaged (which would increase the amount of sample size required), and/or backlighting the filters.

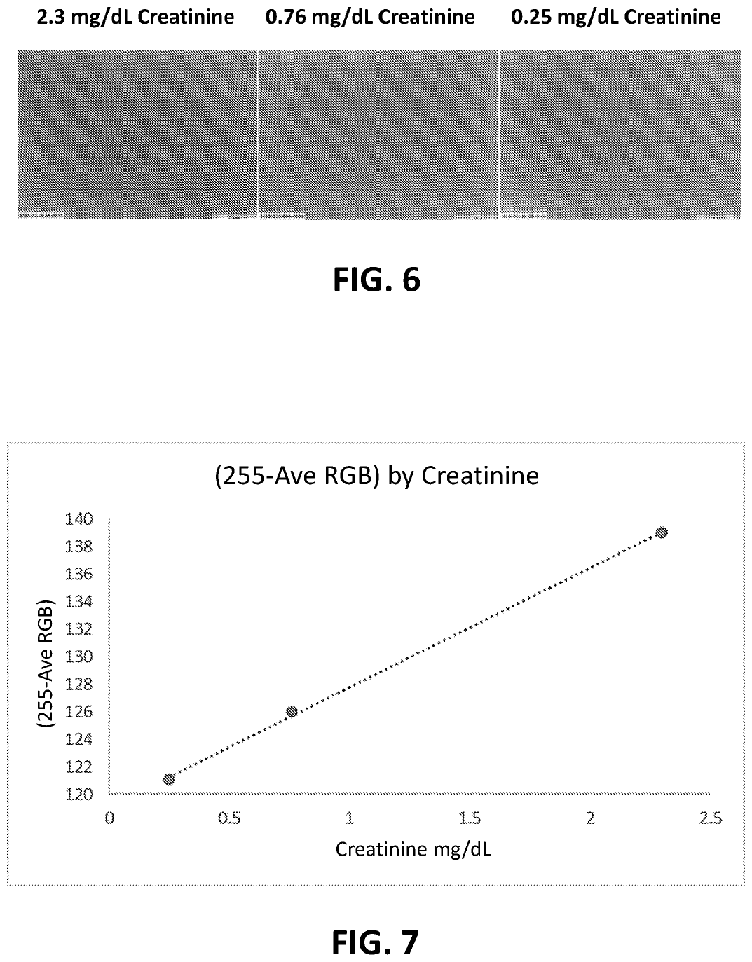

FIGS. 6-8 describe the analysis of the use of 3,5-DNBA and NaOH as colorimetric reagents for the detection of creatinine in the lateral flow assay devices of the present disclosure.

100 mg of 3,5-DNBA (100 mg) was diluted with 6.7575 mL of buffer to dissolve the reagent, and pH was adjusted to 8.8 during this process. There wasn't any perceptible color change at this point with the addition of 10 μL 2.3 mg/dl creatinine solution. Therefore, a dilution series of 50, 25, 12.5, 6, 3, and 0.78% NaOH solution was applied to the pads. When the 10 μL of solution was added, all concentrations except 0.78% triggered color change. Three applications of 10 μL of 0.78% NaOH was the most that could be added before color change was observed. The remaining 3,5-DNBA embedded pads had three applications of 0.78% NaOH applied and dried before 10 μL of creatinine solution (2.3 mg/dL, 0.76 mg/dL, or 0.25 mg/dL) was added to each pad. The pads were mostly dried before being fixed between two acrylic plates, and images of the pads are shown in FIG. 6. The images were taken with a MicroVu; as lighting from the top doesn't differentiate very well visually, a back light was used for the images shown in FIG. 6.

Analysis on these images was then performed. As shown in Table 1 and FIG. 7, the closer to white the color, the closer the values will be to 255. As the creatinine concentration was increased, the darker the pads became. The values were pulled from the entire image averages. These results demonstrate that 3,5-DNBA and NaOH can be utilized in the lateral flow assay devices described or otherwise contemplated herein in the detection of the concentration of creatinine present in a biological fluid sample.

TABLE 1

| Concentration | 2.3 mg/dL | 0.76 mg/dL | 0.25 mg/dL |
|---|---|---|---|
| Red | 114 | 123 | 126 |
| Blue | 136 | 156 | 164 |
| Green | 98 | 108 | 112 |
| Total | 348 | 387 | 402 |
| 255-Ave | 139 | 126 | 121 |

Thus, in accordance with the present disclosure, there have been provided compositions, devices, and kits, as well as methods of producing and using same, which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. A lateral flow assay device for detection of creatinine in a biological fluid sample, the lateral flow assay device comprising:
a housing comprising an upper surface, a lower surface, a first end, a second end, and an interior space extending between the upper and lower surfaces and the first and second ends, the housing further comprising at least a first well and a second well that each extend through the upper surface of the housing into the interior space and that are spaced apart from one another, and wherein the housing further comprises a sample reservoir extending through the upper surface of the housing upstream from the first and second wells;
a lateral flow membrane disposed in the interior space of the housing and defining a path for fluid flow, wherein the lateral flow membrane is in fluidic communication with the sample reservoir and the first and second wells, and wherein the lateral flow membrane has an upper surface and a lower surface;
a first filter membrane disposed in the first well of the housing and on a portion of the upper surface of the lateral flow membrane such that the first filter membrane is in fluidic communication with the lateral flow membrane;
at least one assay reagent for detection of creatinine, wherein the at least one assay reagent is embedded in the first filter membrane;
a second filter membrane disposed in the second well of the housing and on a portion of the upper surface of the lateral flow membrane such that the second filter membrane is in fluidic communication with the lateral flow membrane; and
wherein creatinine is detected in the first well, and hemolysis is detected in the second well.

2. The lateral flow assay device of claim 1, wherein the at least one assay reagent is a colorimetric reagent.

3. The lateral flow assay device of claim 1, wherein the at least one assay reagent comprises 3,5-dinitrobenzoic acid and sodium hydroxide.

4. The lateral flow assay device of claim 1, wherein at least one of the first and second filter membranes comprises an asymmetric cellulose filter membrane.

5. The lateral flow assay device of claim 1, wherein at least one of the first and second filter membranes comprises red blood cell (RBC) agglutinating protein embedded therein.

6. The lateral flow assay device of claim 1, further comprising at least one additional filter membrane disposed in the interior space of the housing downstream of the second well and in fluidic communication with the lateral flow membrane, wherein the at least one additional filter membrane absorbs excess sample.

7. The lateral flow assay device of claim 1, further defined as a lateral flow cartridge configured for insertion within a clinical analyzer instrument.

8. A lateral flow assay device for detection of creatinine in a biological fluid sample, the lateral flow assay device comprising:
a housing comprising an upper surface, a lower surface, a first end, a second end, and an interior space extending between the upper and lower surfaces and the first and second ends, the housing further comprising at least a first well, a second well, and a third well that each extend through the upper surface of the housing into the interior space and that are spaced apart from one another, and wherein the housing further comprises a sample reservoir extending through the upper surface of the housing upstream from the first, second, and third wells;
a lateral flow membrane disposed in the interior space of the housing and defining a path for fluid flow, wherein the lateral flow membrane is in fluidic communication with the sample reservoir and the first, second, and third wells, and wherein the lateral flow membrane has an upper surface and a lower surface;
a first filter membrane disposed in the first well of the housing and on a portion of the upper surface of the lateral flow membrane such that the first filter membrane is in fluidic communication with the lateral flow membrane;
a second filter membrane disposed in the second well of the housing and on a portion of the upper surface of the lateral flow membrane such that the second filter membrane is in fluidic communication with the lateral flow membrane;
at least one assay reagent for detection of creatinine, wherein the at least one assay reagent is embedded in the second filter membrane;
a third filter membrane disposed in the third well of the housing and on a portion of the upper surface of the lateral flow membrane such that the third filter membrane is in fluidic communication with the lateral flow membrane; and
wherein a hematocrit measurement is detected in the first well, creatinine is detected in the second well, and hemolysis is detected in the third well.

9. The lateral flow assay device of claim 8, wherein the at least one assay reagent is a colorimetric reagent.

10. The lateral flow assay device of claim 8, wherein the at least one assay reagent comprises 3,5-dinitrobenzoic acid and sodium hydroxide.

11. The lateral flow assay device of claim 8, wherein at least one of the first, second, and third filter membranes comprises an asymmetric cellulose filter membrane.

12. The lateral flow assay device of claim 8, wherein at least one of the first, second, and third filter membranes comprises red blood cell (RBC) agglutinating protein embedded therein.

13. The lateral flow assay device of claim 8, further comprising at least one additional filter membrane disposed in the interior space of the housing downstream of the third well and in fluidic communication with the lateral flow membrane, wherein the at least one additional filter membrane absorbs excess sample.

14. The lateral flow assay device of claim 8, further defined as a lateral flow cartridge configured for insertion within a clinical analyzer instrument.

15. A kit, comprising:
the lateral flow assay device of claim 1; and
an airtight foiled package in which the lateral flow assay device is disposed.

16. A method for detection of creatinine in a biological fluid sample, the method comprising the steps of:
(1) dispensing the biological fluid sample into the sample reservoir of the lateral flow assay device of claim 1;
(2) allowing the biological fluid sample to flow through the lateral flow assay device;
(3) measuring a signal generated in each of the first and second wells of the lateral flow assay device;
(4) detecting the presence and/or concentration of creatinine based on the signal generated in the first well; and
(5) determining if hemolysis is present based on the signal generated in the second well.

17. The method of claim 16, further comprising the step of inserting the lateral flow assay device into a clinical analyzer instrument after step (1), wherein steps (3), (4), and (5) are performed by the clinical analyzer instrument.

18. A method for detection of creatinine in a biological fluid sample, the method comprising the steps of:
(1) dispensing the biological fluid sample into the sample reservoir of the lateral flow assay device of claim 8;
(2) allowing the biological fluid sample to flow through the lateral flow assay device;
(3) measuring a signal generated in each of the first, second, and third wells of the lateral flow assay device;
(4) detecting a hematocrit value based on the signal generated in the first well;
(5) detecting the presence and/or concentration of creatinine based on the signal generated in the second well; and
(6) determining if hemolysis is present based on the signal generated in the third well.

19. The method of claim 18, further comprising the step of inserting the lateral flow assay device into a clinical analyzer instrument after step (1), wherein steps (3), (4), (5), and (6) are performed by the clinical analyzer instrument.
